(19) United States
(12) Patent Application Publication
(10) Pub. No.: US 2006/0079840 A1
(43) Pub. Date: Apr. 13, 2006

(54) Method and apparatus for controlling the speed of retraction of an automatically retracting needle

(76) Inventor: Edward F. Allard, Springfield, VA (US)

Correspondence Address: Edwaqrd F. Allard, 7830 Greeley Blvd., Springfield, VA, 22152, US

(21) Appl. No.: 10/961,585

(22) Filed: Oct. 12, 2004

(51) Int. Cl. A61M 5/00

(52) U.S. Cl. 604/110; 604/194; 128/919

(57) ABSTRACT

A hypodermic syringe having an automatically retractable needle assembly, releasably activated upon voiding the syringe of injection fluid, with means for controlling the speed of retraction of the retracting needle upon activation of the needle assembly releasing mechanism, allowing the needle to be retracted into the barrel of the plunger for storage.

### BACKGROUND OF THE INVENTION

1. Field of the Invention

The present invention generally relates to automatically retractable hypodermic syringes and blood taking devices, and more specifically to a method and apparatus for controlling the speed of retraction of an automatically retractable hypodermic needle.

2. Description of the Prior Art

Due to the advent of AIDS and other blood transmittable diseases, it has become extremely important to find a way to safely dispose of hypodermic needles and needles from blood taking devices, such as those shown in U.S. Pat. No. 4,838,869, directed to a retractable needle syringe, and U.S. Pat. No. 4,838.863, directed to a retractable needle used in collecting body fluids, both having issued to the same inventor hereof.

In some environments, it has been found desirable to control the speed of retraction of the needle during the retraction process. It is not believed that this particular problem has been previously addressed. Some specific issues that must be considered in solving this problem are the frictional forces being exerted on the needle, the mass of the moving parts, and the friction of flesh on the needle as the needle is removed from the flesh of the patient. One must also consider the effect on the speed of the needle as it tears through the diaphragm of the plunger during the retraction process, keeping in mind that the retracting needle must provide enough force to rupture the diaphragm of the plunger.

### SUMMARY OF THE INVENTION

It is therefore a primary object of the present invention to provide a method and apparatus for controlling the retraction speed of a needle during the period when the needle is being automatically retracted from the flesh of a patient.

Several techniques for accomplishing this purpose are described herein, to include the use of a conical spring, an o-ring or the addition of mass to the needle mechanism to control the speed of retraction of the needle.

### BRIEF DESCRIPTION OF THE DRAWINGS

The foregoing and other objects, aspects and advantages will be better understood from the following detailed description of a preferred embodiment of the invention with reference to the drawings, in which:

FIG. 1 is a cutaway view of one embodiment of an automatically retractable needle assembly; and

FIG. 2 shows an embodiment of the needle assembly of FIG. 1, further having means for controlling the speed of retraction of the needle.

### DETAILED DESCRIPTION OF A PREFERRED EMBODIMENT OF THE INVENTION

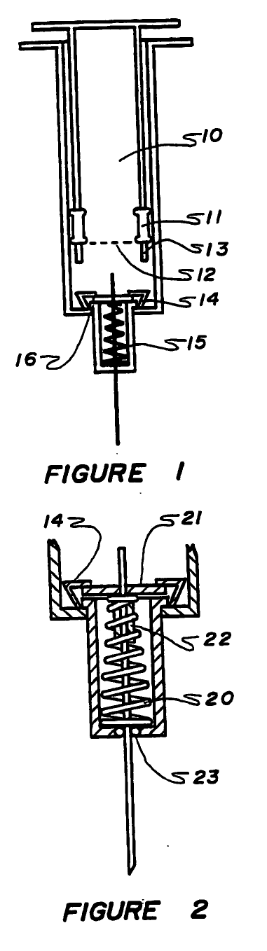

Referring now to the drawings, and more particularly to FIG. 1, which shows a hypodermic needle, similar to that shown in U.S. Pat. No. 4,838,869, wherein a plunger 10 is located inside a hypodermic syringe with a diaphragm 12 attached to the lower end with a slidable seal 11 engaging the inner wall of the syringe. The plunger also has an extension of the plunger wall extending past the diaphragm for contacting release clamps 14, which maintains the spring loaded needle assemble 15 in a readily usable state by engaging a lip 16 of the needle assembly 15, whereby upon termination of the injection process the plunger extension 13 contacts the clamps 14 for releasing the spring loaded needle for irretrievable storage within the plunger housing.

Now looking at FIG. 2, three separate solutions to controlling the speed of the needle assembly 15 of FIG. 1 are depicted, whereby each can be used independently or in any combination.

As is obvious from FIG. 1, upon release of the clamps 14 holding the needle assembly 15, the assembly will experience an initial sudden movement that accelerates the retraction of the needle assembly and this sudden acceleration can be reduced by using a variable force spring 20, added mass 22 or friction o-rings 23, independently or in any combination.

Looking specifically at the variable force spring 20, a conical spring will meet the necessary criteria and will produce a lower force at the beginning of movement and then increase the force as the spring expands, whereas the helical spring will produce a constant force during its expansion. Using a conical spring for control of the needle movement would apply less of a force at the beginning of movement and consequently reduce the acceleration at the onset of motion. This would accordingly reduce the possibility of any damage to the flesh upon retraction of the needle.

Without enumerating the specific calculations, it is well known that a differential equation representing the motion of a needle retracting from the flesh of a human body can be solved analytically, and shows that the speed of retraction depends directly on the frictional forces of the needle rubbing against the o-ring 23, the mass of the moving parts, and the friction due to the needle pulling away from flesh. In reference to the o-ring type seal, applying more pressure on the needle by using an o-ring having a smaller internal diameter increases friction and consequently slows down the moving needle.

It is possible to apply a pressure that will prevent any retraction, or apply a slight pressure that hardly hampers speed at all. Also, it is possible to reduce spring forces by selecting a weaker spring in order to slow down the needle but this is not a solution for some situations. In the case of the retracting needle assembly, the needle must move fast enough to tear through the diaphragm on the plunger. After tearing through the diaphragm the needle still must be pulled from the human body. The energy in the compressed spring is used to overcome friction forces, the body friction force, the work of tearing through the diaphragm, and to transfer kinetic energy to the needle assembly. Using the seal as a brake slows down the needle but allows the needle to be withdrawn completely.

The expression for needle movement shows that increasing the mass of the moving parts can be used to slow down the movement. The kinetic energy of the moving parts is given by KE=mv²/2. Clearly, increasing the mass by a factor of say 4, decreases the velocity (speed) by a factor of two. The needles mass can be increased by using a cylindrical mass 22 attached to the needle.

While the invention has been described in terms of a single preferred embodiment, those skilled in the art will recognize that the invention can be practiced with modification within the spirit and scope of the appended claims.

Having thus described my invention, what I claim as new and desire to secure by Letters Patent is as follows:

1. In a hypodermic syringe:
a fluid holding container;
an actuable plunger within said fluid holding container for forcing the evacuation of any fluids contained therein;
an automatically retractable spring loaded needle assembly attached to the distal end of the fluid holding container;
a rupturable diaphragm sealing the innermost end of the plunger, whereby upon release of the retractable needle assembly, the proximal end of the needle ruptures the diaphragm and the needle is projected inside the plunger for storage; and
means for controlling the speed of retraction of the retracting needle comprising a compressed conical spring, coupled to the retractable needle, which exhibits an increase in force as it propels the needle into the plunger for storage, whereby the speed of the needle is less at the initial release thereof than when the spring is fully extended.

2. The hypodermic syringe of claim 1, wherein the means for controlling the speed of retraction of the retracting needle includes an o-ring integrally mounted within the needle assembly at the distal end of thereof, for passage of the distal end of the needle therethrough, whereby the internal diameter of the o-ring may be selected to provide a frictional force to the shaft of the needle.

3. The hypodermic syringe of claim 2, wherein the means for controlling the speed of retraction of the retracting needle includes an additional mass connected to the needle to absorb some of the kinetic energy stored in the compressed spring, thereby reducing the speed of the retracting needle.

4. A method of controlling the sped of retraction of an automatically retracting needle, including the steps of:
actuating a plunger within a fluid holding container;
expelling the fluid from the fluid holding container;
releasing the retractable needle from a spring loaded needle assembly for storage in the actuating plunger upon contact with the distal end of the actuating plunger and a releasable engagement means functioning to hold said retractable needle in a state of equilibrium within said spring loaded needle assembly;
controlling the speed of retraction of the retracting needle upon release from the spring loaded needle assembly, including the step of providing a conical spring for maintaining the needle in the spring loaded assembly, which spring exhibits the characteristic of an increasing retracting force after the initial release of the spring loaded needle.

5. The method set forth in claim 4, wherein the step of controlling the speed of retraction of the retracting needle further includes the step of providing an o-ring within the distal end of the spring loaded needle assembly for applying radial force to the needle to further slow and control the speed of retraction of the needle.
